(19) United States
(12) Patent Application Publication
(10) Pub. No.: US 2023/0099654 A1
(43) Pub. Date: Mar. 30, 2023

(54) Splittable Sealing Modules for Insertion Assemblies of Rapidly Insertable Central Catheters and Methods Thereof

(71) Applicant: Bard Access Systems, Inc., Salt Lake City, UT (US)

(72) Inventors: Daniel B. Blanchard, Bountiful, UT (US); Glade H. Howell, Draper, UT (US); Kyle G. Thornley, Farmington, UT (US); Eric W. Lindekugel, Salt Lake City, UT (US); Joe Spataro, Cottonwood Heights, UT (US)

(21) Appl. No.: 17/953,860

(22) Filed: Sep. 27, 2022

Related U.S. Application Data
(60) Provisional application No. 63/290,056, filed on Dec. 15, 2021.
(60) Provisional application No. 63/271,043, filed on Oct. 22, 2021.
(60) Provisional application No. 63/249,009, filed on Sep. 27, 2021.

(51) Int. Cl. A61M 25/01; A61M 39/02

(52) U.S. Cl. CPC A61M 25/01; A61M 39/02; A61M 2039/0633

(57) ABSTRACT

Disclosed are splittable sealing modules for insertion assemblies of rapidly insertable central catheters (“RICCs”) and methods thereof. A RICC insertion assembly can include a RICC, an introducer needle, an access guidewire, and a coupler coupling the RICC and the introducer needle together. The introducer needle can include a proximal portion of a sealing-module insert coupled to a distal portion of a needle hub. The coupler can include a coupler housing including a sealing-module cavity and a distal portion of the sealing-module insert disposed in the sealing-module cavity. The sealing-module cavity and the proximal and distal portions of the sealing-module insert form a splittable sealing module of the RICC insertion assembly. The splittable sealing module can be configured to separately seal around the introducer needle and the access guidewire disposed therein when the proximal and distal portions of the sealing-module insert are compressed in the sealing-module cavity.

### PRIORITY

This application claims the benefit of priority to U.S. Provisional Application No. 63/249,009, filed Sep. 27, 2021; U.S. Provisional Application No. 63/271,043, filed Oct. 22, 2021; and U.S. Provisional Application No. 63/290,056, filed Dec. 15, 2021, each of which is incorporated by reference in its entirety into this application.

### BACKGROUND

Central venous catheters (“CVCs”) are commonly introduced into patients and advanced through their vasculatures by way of the Seldinger technique. The Seldinger technique utilizes a number of steps and medical devices (e.g., a needle, a scalpel, a guidewire, an introducer sheath, a dilator, a CVC, etc.). While the Seldinger technique is effective, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are insertion assemblies of rapidly insertable central catheters (“RICCs”) and methods that address the foregoing. Notably, the RICC insertion assemblies disclosed herein include splittable sealing modules for sealing different components of the RICC insertion assemblies therein.

### SUMMARY

Disclosed herein is a RICC insertion assembly including, in some embodiments, a RICC, an introducer needle, an access guidewire, and a coupler coupling the RICC and the introducer needle together. The RICC includes a catheter tube and a primary lumen therethrough. The introducer needle includes a needle shaft, a needle hub around a proximal-end portion of the needle shaft, and a proximal portion of a sealing-module insert coupled to a distal portion of the needle hub. The access guidewire includes a proximal portion disposed in the primary lumen of the RICC and a distal portion disposed in the needle shaft through a longitudinal needle slot of the needle shaft. The coupler includes a coupler housing including a sealing-module cavity and a distal portion of the sealing-module insert disposed in the sealing-module cavity. The sealing-module cavity and the proximal and distal portions of the sealing-module insert form a splittable sealing module of the RICC insertion assembly. The splittable sealing module is configured to separately seal around a proximal portion of the introducer needle and the distal portion of the access guidewire when the proximal and distal portions of the sealing-module insert are compressed in the sealing-module cavity.

In some embodiments, each portion of the proximal and distal portions of the sealing-module insert is formed of an elastomer.

In some embodiments, the proximal and distal portions of the sealing-module insert complete both an introducer-needle passageway and an access-guidewire passageway as combined in the sealing-module cavity.

In some embodiments, a proximal portion of the introducer-needle passageway is present in the proximal portion of the sealing-module insert. In addition, a distal portion of the introducer-needle passageway is completed by the proximal and distal portions of the sealing-module insert combined in the sealing-module cavity.

In some embodiments, an entirety of the access-guidewire passageway is completed by the proximal and distal portions of the sealing-module insert combined in the sealing-module cavity.

In some embodiments, a distal end of the access-guidewire passageway connects to a medial portion of the introducer-needle passageway. Such a connection allows the distal portion of the access guidewire to be disposed in the needle shaft through the needle slot.

In some embodiments, the introducer-needle passageway is configured to seal around the proximal portion of the introducer needle when the proximal and distal portions of the sealing-module insert are compressed in the sealing-module cavity. In addition, the access-guidewire passageway is configured to seal around the distal portion of the access guidewire when the proximal and distal portions of the sealing-module insert are compressed in the sealing-module cavity.

In some embodiments, the introducer-needle passageway includes an internal relief between proximal and distal portions of the introducer-needle passageway such that only proximal- and distal-end portions of the introducer-needle passageway are configured to seal around the proximal portion of the introducer needle when the proximal and distal portions of the sealing-module insert are compressed in the sealing-module cavity.

In some embodiments, the access-guidewire passageway includes an internal relief in at least a proximal portion of the access-guidewire passageway such that only a proximal-end portion of the access-guidewire passageway is configured to seal around the distal portion of the access guidewire when the proximal and distal portions of the sealing-module insert are compressed in the sealing-module cavity.

In some embodiments, the sealing-module insert is radially compressed in the sealing-module cavity by the coupler housing.

In some embodiments, the sealing-module insert is axially compressed in the sealing-module cavity by the distal portion of the needle hub. Axial compression of the sealing-module insert in the sealing-module cavity, in turn, radially compresses the sealing-module insert in the sealing-module cavity.

In some embodiments, the coupler housing includes a longitudinal coupler-housing slot configured to allow the access guidewire to escape from the coupler housing after the proximal portion of the sealing-module insert is removed from the sealing-module cavity with withdrawal of the introducer needle from the coupler.

In some embodiments, the introducer needle further includes a sheath over the needle shaft sealing the needle slot thereunder. The needle slot extends from a proximal portion of the needle shaft through a distal needle tip.

In some embodiments, the coupler includes a blade extending into the splittable sealing module such that the blade is disposed in the needle slot under a distal end of a sheath opening. The blade includes a distal-facing blade edge configured to cut the sheath away from the needle shaft as the introducer needle is withdrawn from the coupler. Cutting the sheath away from the needle shaft allows the access guidewire to escape from the needle shaft by way of the needle slot.

In some embodiments, the blade is overmolded into the coupler housing, thereby integrating the blade in the coupler housing.

In some embodiments, the sheath is splittable.

In some embodiments, the sheath is perforated with a pattern of holes, slits, or a combination thereof longitudinally extending over the sheath configured for splitting the sheath. The pattern is offset from the needle slot to maintain the sealing of the needle slot by the sheath.

In some embodiments, the sheath includes one or more grooves longitudinally extending over the sheath configured for splitting the sheath.

In some embodiments, the sheath includes an embedded pull cord longitudinally extending within the sheath configured for splitting the sheath when pulled away from the sheath.

In some embodiments, the sheath is formed of a polymeric material selected from polyethylene, polypropylene, polyurethane, and polytetrafluoroethylene.

In some embodiments, the coupler further includes an access guidewire-connecting side arm. The access guidewire-connecting side arm includes a connector configured to connect to an access-guidewire hub about a proximal-end portion of the access guidewire. The distal portion of the access guidewire is disposed in the needle shaft and the access-guidewire hub is connected to the connector of the access guidewire-connecting side arm, thereby enforcing a loop in the access guidewire over which loop the RICC is disposed in at least a ready-to-operate state of the RICC insertion assembly.

In some embodiments, the RICC insertion assembly further includes a keeper. The keeper includes a splittable casing over both the catheter tube of the RICC and an extracatheteral portion of the access guidewire extending from a distal end of the RICC. The splittable casing is configured to keep the catheter tube and the extracatheteral portion of the access guidewire sterile until deployed.

In some embodiments, the keeper further includes a catheter-hub holder to which a proximal end of the splittable casing is attached. The catheter-hub holder is configured to hold a catheter hub of the RICC therein and keep the splittable casing in position over both the catheter tube and the extracatheteral portion of the access guidewire.

In some embodiments, the catheter-hub holder includes a perimetrical wall around at least a portion of a perimeter of the catheter-hub holder. The perimetrical wall defines a recess into which the catheter hub fits with an engineering fit.

In some embodiments, the coupler further includes a splittable casing-holding side arm including a primary channel and a secondary channel. The primary channel is configured to slidably hold the splittable casing therein. The secondary channel is configured to guide the access guidewire split away from the splittable casing into the coupler and the needle shaft through the needle slot.

In some embodiments, the coupler further includes an access guidewire-connecting side arm. The access guidewire-connecting side arm includes a connector configured to connect to an access-guidewire hub about a proximal-end portion of the access guidewire. The distal portion of the access guidewire is disposed in the needle shaft, a distal portion of the splittable casing is held in the primary channel of the splittable casing-holding side arm, and the access-guidewire hub is connected to the connector of the access guidewire-connecting side arm, thereby enforcing a loop in the access guidewire over which loop the RICC is disposed in at least a ready-to-operate state of the RICC insertion assembly.

In some embodiments, the RICC includes a set of three lumens including the primary lumen, a secondary lumen, and a tertiary lumen. The set of three lumens are formed of fluidly connected portions of three catheter-tube lumens, three catheter-hub lumens, and three extension-leg lumens.

In some embodiments, the primary lumen has a primary-lumen aperture in a distal end of the catheter tube, the secondary lumen has a secondary-lumen aperture in a side of a distal portion of catheter tube, and the tertiary lumen has a tertiary-lumen aperture in the side of the distal portion of the catheter tube proximal of the secondary-lumen aperture.

Also disclosed herein is a method for inserting a RICC into a blood-vessel lumen of a patient. The method includes, in some embodiments, an insertion assembly-obtaining step, a needle tract-establishing step, an access guidewire-advancing step, and a RICC-advancing step. The insertion assembly-obtaining step includes obtaining a RICC insertion assembly, optionally, already in a ready-to-operate state thereof. The RICC insertion assembly includes the RICC, an introducer needle, an access guidewire, and a coupler coupling the RICC and the introducer needle together. In addition, the RICC insertion assembly includes a splittable sealing module formed between at least a sealing-module cavity of a coupler housing of the coupler, a proximal portion of a sealing-module insert coupled to a distal portion of a needle hub of the introducer needle, and a distal portion of the sealing-module insert disposed in the sealing-module cavity. The splittable sealing module separately seals around a proximal portion of the introducer needle and a distal portion of the access guidewire. The needle tract-establishing step includes establishing a needle tract from an area of skin to the blood-vessel lumen with the introducer needle. The access guidewire-advancing step includes advancing the distal portion of the access guidewire through both the splittable sealing module and a longitudinal needle slot of a needle shaft of the introducer needle such that a distal end of the access guidewire is advanced from a location in the introducer needle just proximal of a needle tip of the needle shaft into the blood-vessel lumen. The RICC-advancing step includes advancing a catheter tube of the RICC over the access guidewire and into the blood-vessel lumen, thereby inserting the RICC into the blood-vessel lumen.

In some embodiments, the proximal and distal portions of the sealing-module insert complete both an introducer-needle passageway and an access-guidewire passageway as combined in the sealing-module cavity.

In some embodiments, a proximal portion of the introducer-needle passageway is present in the proximal portion of the sealing-module insert. In addition, a distal portion of the introducer-needle passageway is completed by the proximal and distal portions of the sealing-module insert combined in the sealing-module cavity.

In some embodiments, an entirety of the access-guidewire passageway is completed by the proximal and distal portions of the sealing-module insert combined in the sealing-module cavity.

In some embodiments, a distal end of the access-guidewire passageway connects to a medial portion of the introducer-needle passageway. Such a connection allows the distal portion of the access guidewire to be disposed in the needle shaft through a longitudinal needle slot of the needle shaft.

In some embodiments, the introducer-needle passageway is configured to seal around the proximal portion of the introducer needle when the proximal and distal portions of the sealing-module insert are compressed in the sealing-module cavity. In addition, the access-guidewire passageway is configured to seal around the distal portion of the access guidewire when the proximal and distal portions of the sealing-module insert are compressed in the sealing-module cavity.

In some embodiments, the method further includes a state-confirming step. The state-confirming step includes confirming the RICC insertion assembly is in the ready-to-operate state thereof before the needle tract-establishing step. Such confirmation ensures the proximal and distal portions of the sealing-module insert are compressed in the sealing-module cavity sealing the introducer-needle passageway and the access-guidewire passageway around the introducer needle and access guidewire, respectively.

In some embodiments, the access guidewire-advancing step includes pinching an extracatheteral portion of the access guidewire extending from a distal end of the RICC and pushing the access guidewire into needle shaft via the splittable sealing module of the coupler. Advancing the access guidewire in this way reduces a loop in the access guidewire over which loop the RICC is disposed. The loop is enforced by the distal portion of the access guidewire disposed in the needle shaft and an access-guidewire hub of the access guidewire connected to a connector of an access guidewire-connecting side arm of the coupler.

In some embodiments, the access guidewire-advancing step includes pinching a splittable casing over both the catheter tube of the RICC and an extracatheteral portion of the access guidewire extending from a distal end of the RICC. The access guidewire-advancing step also includes pushing the splittable casing into a primary channel of a splittable casing-holding side arm of the coupler while pinching the splittable casing. With such pinching and pushing of the splittable casing into the primary channel of the splittable casing-holding side arm, the access guidewire splits away from the splittable casing into a secondary channel of the splittable casing-holding side arm and, subsequently, the needle shaft via the splittable sealing module of the coupler.

In some embodiments, the access guidewire-advancing step includes reducing a loop in the access guidewire over which loop the RICC is disposed. The loop is enforced by the splittable casing held in the primary channel of the splittable casing-holding side arm and an access-guidewire hub of the access guidewire connected to a connector of an access guidewire-connecting side arm of the coupler.

In some embodiments, the method further includes an introducer needle-withdrawing step. The introducer needle-withdrawing step includes withdrawing the introducer needle from the coupler leaving the access guidewire in place in the blood-vessel lumen before the RICC-advancing step. The withdrawing of the introducer needle from the coupler removes the needle hub of the introducer needle from a needle-hub receptacle and, with the needle hub, the proximal portion of the sealing-module insert from the sealing-module cavity, thereby splitting the proximal and distal portions of the sealing-module insert away from each other and unsealing the proximal portion of the introducer needle and the distal portion of the access guidewire so the access guidewire is allowed to escape the splittable sealing module.

In some embodiments, the introducer needle-withdrawing step includes cutting a needle slot-sealing sheath of the introducer needle away from the needle shaft. The coupler includes a blade extending into the splittable sealing module such that the blade is disposed in the needle slot under a distal end of a sheath opening of the sheath with a distal-facing blade edge for the cutting of the sheath away from the needle shaft.

In some embodiments, the cutting of the sheath away from the needle shaft allows the access guidewire to escape from the needle shaft by way of the needle slot.

In some embodiments, a longitudinal coupler-housing slot of the coupler housing allows the access guidewire to escape from the coupler housing after the introducer needle-withdrawing step.

In some embodiments, the needle tract-establishing step includes ensuring blood flashes back into the introducer needle or a syringe fluidly connected to the introducer needle, thereby confirming the needle tract extends into the blood-vessel lumen.

In some embodiments, the method further includes a blood-aspirating step. The blood-aspirating step includes aspirating blood with the syringe to confirm the needle tract extends into the blood-vessel lumen.

In some embodiments, the method further includes an access guidewire-withdrawing step. The access guidewire-withdrawing step includes withdrawing the access guidewire leaving the catheter tube in place in the blood-vessel lumen.

In some embodiments, the method further includes a maneuver guidewire-advancing step, another RICC-advancing step, and a maneuver guidewire-withdrawing step. The maneuver guidewire-advancing step includes advancing a maneuver guidewire into the blood-vessel lumen by way of a primary lumen of the RICC. The other RICC-advancing step includes advancing the catheter tube farther into the blood-vessel lumen over the maneuver guidewire to a lower ⅓ of a superior vena cava (“SVC”) of a heart of the patient. The maneuver guidewire-withdrawing step includes withdrawing the maneuver guidewire leaving the catheter tube in place in the lower ⅓ of the SVC.

These and other features of the concepts provided herein will become more apparent to those of skill in the art in view of the accompanying drawings and following description, which describe particular embodiments of such concepts in greater detail.

### DRAWINGS

FIG. 1 illustrates a RICC insertion assembly in accordance with some embodiments.

FIG. 2 illustrates a perspective view of a coupler of the RICC insertion assembly in accordance with some embodiments.

FIG. 3 illustrates a side view of the coupler in accordance with some embodiments.

FIG. 4 illustrates a longitudinal cross section of the coupler and a splittable sealing module thereof in accordance with some embodiments.

FIG. 5 illustrates a perspective view of a needle hub an introducer needle and proximal and distal portions of a sealing-module insert of the splittable sealing module in accordance with some embodiments.

FIG. 6 illustrates a longitudinal cross section of the needle hub and the proximal and distal portions of the sealing-module insert in accordance with some embodiments.

FIG. 7 illustrates a longitudinal cross section of the splittable sealing module in two different states in accordance with some embodiments.

FIG. 8 illustrates a transverse cross section of a proximal portion of the splittable sealing module in the two different states in accordance with some embodiments.

FIG. 9 illustrates radial compression of the sealing-module insert in a sealing-module cavity of the splittable sealing module defined by a coupler housing of the coupler in accordance with some embodiments.

FIG. 10 illustrates radial compression of the sealing-module insert in the sealing-module cavity through axial compression of the sealing-module insert in the sealing-module cavity by the needle hub in accordance with some embodiments.

FIG. 11 illustrates a longitudinal cross section of another sealing-module insert with internal reliefs in an introducer-needle passageway and an access-guidewire passageway of the sealing-module insert in accordance with some embodiments.

FIG. 12 illustrates a top view of the introducer needle in accordance with some embodiments.

FIG. 13A illustrates a cuttable sheath of the introducer needle in accordance with some embodiments.

FIG. 13B illustrates a splittable sheath of the introducer needle in accordance with some embodiments.

FIG. 13C illustrates another splittable sheath of the introducer needle in accordance with some embodiments.

FIG. 13D illustrates yet another splittable sheath of the introducer needle in accordance with some embodiments.

FIG. 14 illustrates a needle shaft of the introducer needle in accordance with some embodiments.

FIG. 15 illustrates a RICC of the RICC insertion assembly in accordance with some embodiments.

FIG. 16 illustrates a detailed view of a distal portion of a catheter tube of the RICC in accordance with some embodiments.

FIG. 17 illustrates a transverse cross section of the distal portion of the catheter tube in accordance with some embodiments.

FIG. 18 illustrates another transverse cross section of the distal portion of the catheter tube in accordance with some embodiments.

FIG. 19 illustrates a longitudinal cross section of the distal portion of the catheter tube in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. In addition, any of the foregoing features or steps can, in turn, further include one or more features or steps unless indicated otherwise. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

With respect to “proximal,” a “proximal portion” or a “proximal-end portion” of, for example, a catheter includes a portion of the catheter intended to be near a clinician when the catheter is used on a patient. Likewise, a “proximal length” of, for example, the catheter includes a length of the catheter intended to be near the clinician when the catheter is used on the patient. A “proximal end” of, for example, the catheter includes an end of the catheter intended to be near the clinician when the catheter is used on the patient. The proximal portion, the proximal-end portion, or the proximal length of the catheter can include the proximal end of the catheter; however, the proximal portion, the proximal-end portion, or the proximal length of the catheter need not include the proximal end of the catheter. That is, unless context suggests otherwise, the proximal portion, the proximal-end portion, or the proximal length of the catheter is not a terminal portion or terminal length of the catheter.

With respect to “distal,” a “distal portion” or a “distal-end portion” of, for example, a catheter includes a portion of the catheter intended to be near or in a patient when the catheter is used on the patient. Likewise, a “distal length” of, for example, the catheter includes a length of the catheter intended to be near or in the patient when the catheter is used on the patient. A “distal end” of, for example, the catheter includes an end of the catheter intended to be near or in the patient when the catheter is used on the patient. The distal portion, the distal-end portion, or the distal length of the catheter can include the distal end of the catheter; however, the distal portion, the distal-end portion, or the distal length of the catheter need not include the distal end of the catheter. That is, unless context suggests otherwise, the distal portion, the distal-end portion, or the distal length of the catheter is not a terminal portion or terminal length of the catheter.

Unless defined otherwise, all technical and scientific terms used herein have the same meaning as commonly understood by those of ordinary skill in the art.

As set forth above with respect to the Seldinger technique, the number of steps are time consuming, handling the number of medical devices is awkward, and both of the foregoing can lead to patient trauma. In addition, there is a relatively high potential for touch contamination due to the number of medical devices that need to be interchanged during the Seldinger technique. As such, there is a need to reduce the number of steps and medical devices involved in introducing a catheter such as a CVC into a patient and advancing the catheter through a vasculature thereof.

Disclosed herein are insertion assemblies of RICCs and methods that address the foregoing. Notably, the RICC insertion assemblies disclosed herein include splittable sealing modules for sealing different components of the RICC insertion assemblies therein. For example, a RICC insertion assembly can include a RICC, an introducer needle, an access guidewire, and a coupler coupling the RICC and the introducer needle together. The introducer needle can include a proximal portion of a sealing-module insert coupled to a distal portion of a needle hub. The coupler can include a coupler housing including a sealing-module cavity and a distal portion of the sealing-module insert disposed in the sealing-module cavity. The sealing-module cavity and the proximal and distal portions of the sealing-module insert form a splittable sealing module of the RICC insertion assembly. The splittable sealing module can be configured to separately seal around the introducer needle and the access guidewire disposed therein when the proximal and distal portions of the sealing-module insert are compressed in the sealing-module cavity.

The foregoing features as well as other features of the RICC insertion assemblies and methods disclosed herein will become more apparent to those of skill in the art in view of the accompanying drawings and following description, which describe particular embodiments of the RICC insertion assemblies and methods in greater detail. However, it should be understood the RICCs of the RICC insertion assemblies are but one type of catheter that can be incorporated into catheter insertion assemblies like those disclosed herein. Indeed, peripherally inserted central catheters (“PICCs”), dialysis catheters, or the like can also be incorporated into catheter insertion assemblies and methods like those disclosed herein.

### RICC Insertion Assemblies

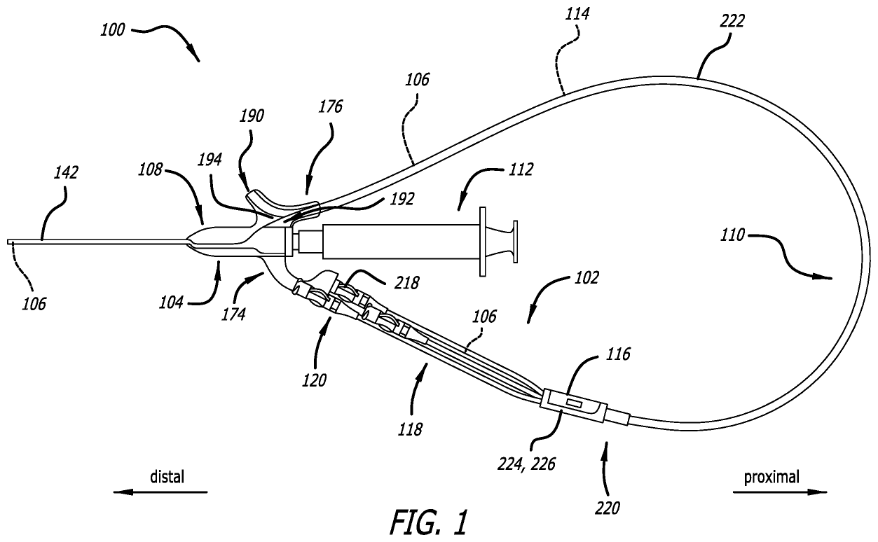

FIG. 1 illustrates a RICC insertion assembly 100 in accordance with some embodiments.

As shown, the RICC insertion assembly 100 includes a RICC 102, an introducer needle 104, an access guidewire 106, and a coupler 108 coupling the RICC 102, the introducer needle 104, and the access guidewire 106 together in a ready-to-operate state of the RICC insertion assembly 100. As set forth in more detail below, the proximal end of the access guidewire 106 is coupled to the coupler 108 and the distal end of the access guidewire 106 is disposed in the needle lumen 158 of the introducer needle 104 in the ready-to-operate state of the RICC insertion assembly 100. This enforces a loop 110 in the access guidewire 106, which loop 110 the RICC 102 is disposed over in the ready-to-operate state of the RICC insertion assembly 100 keeping the RICC insertion assembly 100 in a relatively compact form.

The RICC insertion assembly 100 can further include a syringe 112 fluidly coupled to the introducer needle 104 in the ready-to-operate state of the RICC insertion assembly 100. As set forth below, the splittable sealing module 196 seals around the proximal portion of the introducer needle 104 and the distal portion of the access guidewire 106 when the sealing-module insert 198 is compressed in the sealing-module cavity 178 in one or more states of the RICC insertion assembly 100. In particular, the splittable sealing module 196 seals over the sheath opening 162 of the sheath 142 that opens to the needle slot 150 of the needle shaft 144. Outside of the splittable sealing module 196, the sheath 142 seals the needle slot 150 of the needle shaft 144. Such seals enable the syringe 112 to aspirate blood in accordance with the blood-aspirating step of the method set forth below.

Lastly, any component of the RICC insertion assembly 100 selected from at least the RICC 102, the introducer needle 104, the access guidewire 106, the coupler 108, and the syringe 110, or any portion of the component selected from the foregoing components, can include an antimicrobial thereon or therein. In an example, the catheter tube 114 of the RICC 102 can include an antimicrobial coating on an abluminal surface of the catheter tube 114, a luminal surface of the catheter tube 114, or both. In another example, a pre-extrusion material of the catheter tube 114 can include the antimicrobial admixed therein such that the antimicrobial is incorporated into the catheter tube 114 when extruded, the antimicrobial protecting both the abluminal surface of the catheter tube 114 and the luminal surface of the catheter tube 114 from microbial contamination.

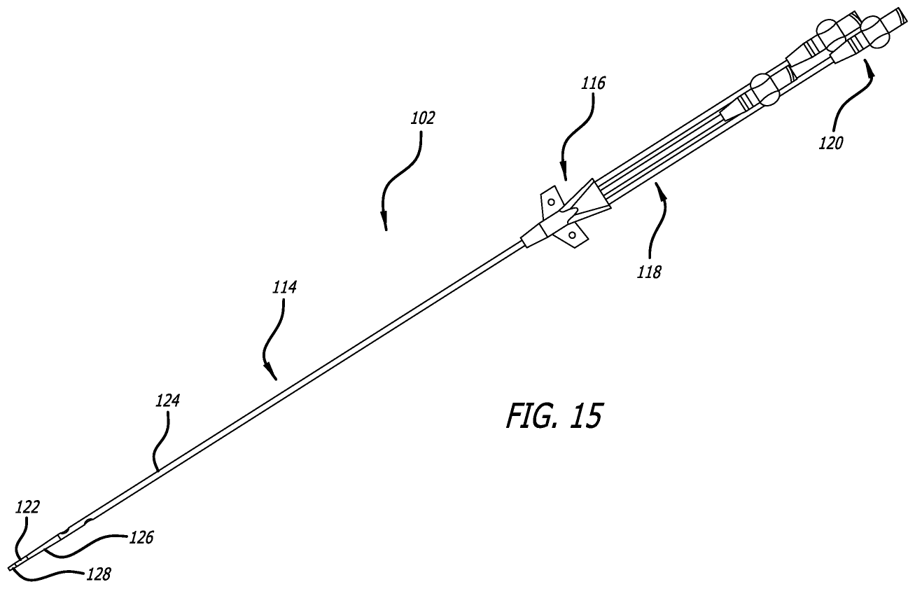

FIG. 15 illustrates the RICC 102 of the RICC insertion assembly 100 in accordance with some embodiments.

As shown, the RICC 102 includes a catheter tube 114, a catheter hub 116, one or more extension legs 118, and one or more extension-leg connectors 120.

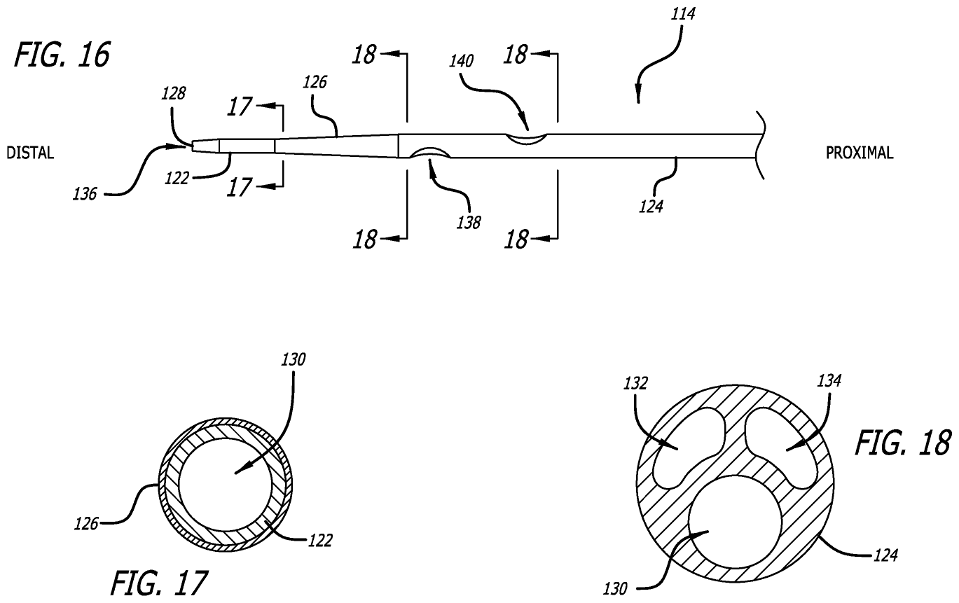

FIGS. 16-19 illustrate various views of the catheter tube 114 of the RICC 102 in accordance with some embodiments.

The catheter tube 114 includes a first section 122 in a distal portion of the catheter tube 114, a second section 124 in the distal portion of the catheter tube 114 proximal of the first section 122, and a tapered junction 126 between the first and second sections 122 and 124 of the catheter tube 114.

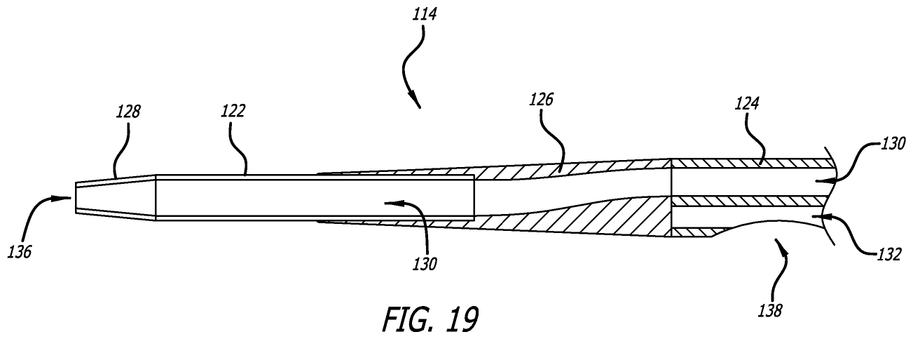

The first section 122 of the catheter tube 114 includes a catheter tip 128 having a relatively short taper from an outer diameter of a distal portion of the first section 122 distal of the junction 126 to an outer diameter of a distal end of the first section 122. The taper of the catheter tip 128 is configured for immediate dilation of tissue about a needle tract established with the introducer needle 104 up to the outer diameter of the distal portion of the first section 122 of the catheter tube 114. As best shown in FIG. 19, the first section 122 of the catheter tube 114 also includes a proximal portion disposed in a bore of a distal portion of the junction 126 and fixedly coupled thereto such as by a solvent bond, an adhesive bond, or a heat weld.

The second section 124 of the catheter tube 114 includes a consistent outer diameter over its length from a distal end of the second section 124 to a proximal end of the second section 124. The consistent diameter of the second section 124 of the catheter tube 114 is configured for smooth insertion into the needle tract and targeted vasculature subsequent to any dilation by the first section 122 of the catheter tube 114 and the junction 126. The distal end of the second section 124 of the catheter tube 114 has a flat face flush with the flat-faced proximal end of the junction 126 and fixedly coupled thereto such as by a solvent bond, an adhesive bond, or a heat weld.

The junction 126 includes a taper over its length from a proximal end of the junction 126 to a distal end of the junction 126. The taper of the junction 126 is configured for immediate dilation of the tissue about the needle tract from the outer diameter of the proximal portion of the first section 122 of the catheter tube 114 to the outer diameter of the second section 124 of the catheter tube 114. An abluminal surface of the junction 126 smoothly transitions from an abluminal surface of the first section 122 of the catheter tube 114 to an abluminal surface of the second section 124 of the catheter tube 114 without edges that catch on skin when the catheter tube 114 is inserted into the needle tract. In addition to the edges being minimal to negligible, the edges can include solvent-interdiffused polymeric material of the polymeric materials from which the catheter tube 114 is formed, which smoothens the transitions from the first section 122 of the catheter tube 114 to the junction 126 and from the junction 126 to the second section 124 of the catheter tube 114. Notably, the junction 126 has a length approximately commensurate with a length of an exposed portion of the first section 122 of the catheter tube 114 or between lengths of exposed portions of the first and second sections 122 and 124 of the catheter tube 114. As such, the length of the exposed portion of the first section 122 of the catheter tube 114 is less than the length of the junction 126 up to approximately commensurate with the length of the junction 126.

The first section 122 of the catheter tube 114 is formed of a first polymeric material (e.g., a polytetrafluoroethylene, a polypropylene, or a polyurethane) having a first durometer. The second section 124 of the catheter tube 114 is formed of a second polymeric material (e.g., a polyvinyl chloride, a polyethylene, another polyurethane, or a silicone) having a second durometer less than the first durometer. For example, the first section 122 of the catheter tube 114 can be formed of a first polyurethane having the first durometer while the second section 124 of the catheter tube 114 can be formed of a second, different polyurethane (e.g., a same or different diisocyanate or triisocyanate reacted with a different diol or triol, a different diisocyanate or triisocyanate reacted with a same or different diol or triol, a same diisocyanate or triisocyanate reacted with a same diol or triol under different conditions or with different additives, etc.) having the second durometer less than the first durometer. Indeed, polyurethanes are advantageous for the catheter tube 114 in that polyurethanes can be relatively rigid at room-temperature but become more flexible in vivo at body temperature, which reduces irritation to vessel walls as well as phlebitis. Polyurethanes are also advantageous in that they can be less thrombogenic than some other polymers. The junction 126 is formed of the second polymeric material or a third polymeric material (e.g., yet another polyurethane) having a third durometer less than the first durometer and greater than, approximately equal to, or less than the second durometer.

It should be understood the first durometer of the first polymeric material, the second durometer of the second polymeric material, and the third durometer of the third polymeric material can be on different scales (e.g., Type A or Type D). With this understanding, the second durometer of the second polymeric material or the third durometer of the third polymeric material might not be numerically less than the first durometer of the first polymeric material when the second durometer or the third durometer is less than the first durometer. Indeed, the hardness of the second polymeric material or the third polymeric material can still be less than the hardness of the first polymeric material as the different scales—each of which ranges from 0 to 100—are designed for characterizing different materials in groups of the materials having a like hardness.

In accordance with the first section 122 of the catheter tube 114, the second section 124 of the catheter tube 114, and the junction 126 between the first and second sections 122 and 124 of the catheter tube 114 set forth above, the catheter tube 114 possesses a column strength sufficient to prevent buckling of the catheter tube 114 when inserted into a needle tract established by with the introducer needle 104. The column strength of the catheter tube 114 is also sufficient to prevent buckling of the catheter tube 114 when advanced through a vasculature of a patient without dilation of tissue about the needle tract or any blood vessels of the vasculature beforehand with a separate dilator.

The catheter tube 114 includes one or more catheter-tube lumens extending through the catheter tube 114; however, only one catheter-tube lumen typically extends from a proximal end of the catheter tube 114 to a distal end of the catheter tube 114 in a multiluminal RICC (e.g., a diluminal RICC, a triluminal RICC, a tetraluminal RICC, a pentaluminal RICC, a hexaluminal RICC, etc.). (See FIGS. 16-19.) Indeed, the first section 122 of the catheter tube 114 typically includes a single lumen therethrough as shown in FIGS. 17 and 19.

The catheter hub 116 is coupled to a proximal portion of the catheter tube 114. The catheter hub 116 includes one or more catheter-hub lumens corresponding in number to the one-or-more catheter-tube lumens. The one-or-more catheter-hub lumens extends through an entirety of the catheter hub 116 from a proximal end of the catheter hub 116 to a distal end of the catheter hub 116.

Each extension leg of the one-or-more extension legs 118 is coupled to the catheter hub 116 by a distal portion thereof. The one-or-more extension legs 118 respectively include one or more extension-leg lumens, which, in turn, correspond in number to the one-or-more catheter-hub lumens. Each extension-leg lumen of the one-or-more extension-leg lumens extends through an entirety of the extension leg from a proximal end of the extension leg to a distal end of the extension leg.

Each extension-leg connector of the one-or-more extension-leg connectors 120 is over a proximal portion of an extension leg of the one-or-more extension legs 118. For example, each extension-leg connector of the one-or-more extension-leg connectors 120 can be a Luer connector over a proximal portion of an extension leg of the one-or-more extension legs 118. Through such an extension-leg connector, a corresponding extension leg and the extension-leg lumen thereof can be connected to another medical device and a lumen thereof. However, in the ready-to-operate state of the RICC insertion assembly 100 at least one extension-leg connector (e.g., the extension-leg connector including part of the primary lumen 130 of the RICC 102) is indirectly connected via the intervening access-guidewire hub 218 to the access guidewire-connecting side arm 174 of the coupler 108 to enforce the loop 110 in the access guidewire 106 and the RICC 102 thereover.

As shown, the RICC 102 is a triluminal RICC including a set of three lumens; however, the RICC 102 is not limited to the set of the three lumens as set forth above. The set of three lumens includes a primary lumen 130, a secondary lumen 132, and a tertiary lumen 134 formed of fluidly connected portions of three catheter-tube lumens, three catheter-hub lumens, and three extension-leg lumens. The primary lumen 130 has a primary-lumen aperture 136 in the distal end of the first section 122 of the catheter tube 114, which corresponds to the distal end of the catheter tube 114 and a distal end of the RICC 102. The secondary lumen 132 has a secondary-lumen aperture 138 in a side of the distal portion of the catheter tube 114. The tertiary lumen 134 has a tertiary-lumen aperture 140 in the side of the distal portion of the catheter tube 114 proximal of the secondary-lumen aperture 138.

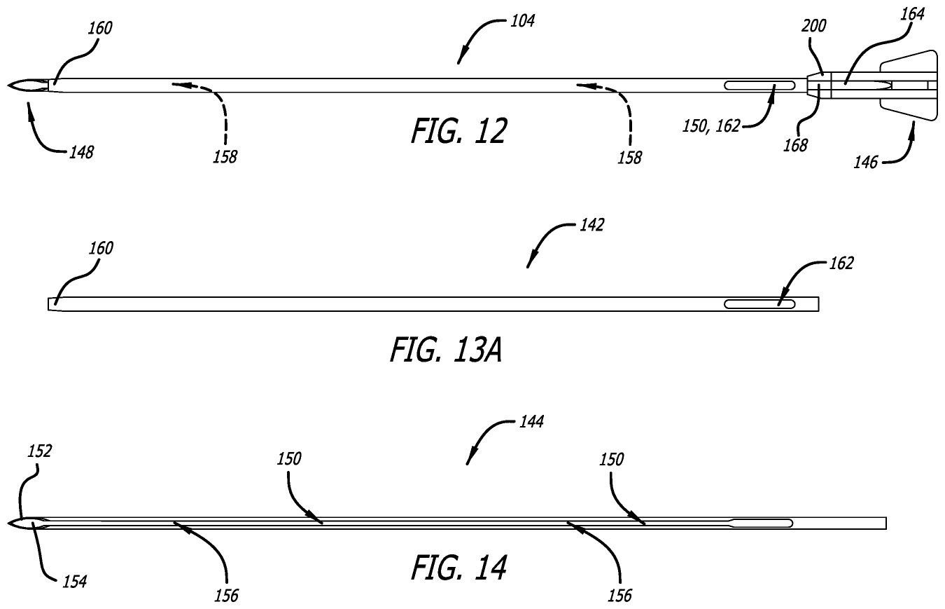

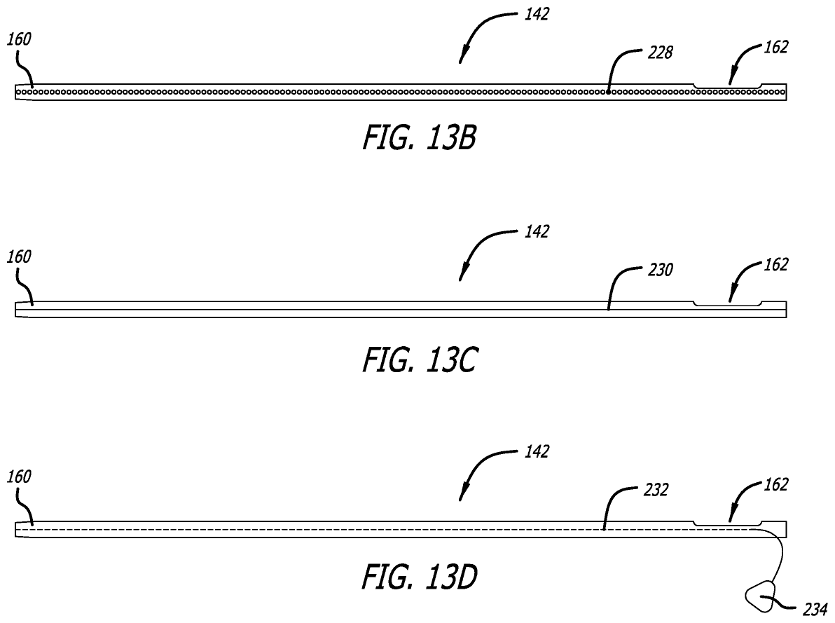

FIG. 12 illustrates a top view of the introducer needle 104 of the RICC insertion assembly 100 in accordance with some embodiments. FIGS. 13A-13D illustrate a sheath 142 of the introducer needle 104 in accordance with some embodiments. FIG. 14 illustrates a needle shaft 144 of the introducer needle 104 in accordance with some embodiments.

As shown, the introducer needle 104 includes the needle shaft 144, the sheath 142 over the needle shaft 144, and a needle hub 146 in a proximal portion of the introducer needle 104 over both a proximal-end portion of the needle shaft 144 and a proximal-end portion of the sheath 142. In addition, the introducer needle 104 can be considered to include the proximal portion 200 of the sealing-module insert 198 coupled to the distal portion of the needle hub 146, particularly when the proximal portion 200 of the sealing-module insert 198 is fixedly, as opposed to removably, coupled to the needle hub 146. In at least the ready-to-operate state of the RICC insertion assembly 100, the needle shaft 144 and the sheath 142 extend from the needle hub 146, through the splittable sealing module 196, and out a distal end of the coupler housing 172.

The needle shaft 144 includes a needle tip 148 in a distal portion of the needle shaft 144 and a longitudinal needle slot 150 extending from the proximal portion of the needle shaft 144 through the needle tip 148.

The needle tip 148 includes a bevel having a tip bevel 152 and a primary bevel 154 proximal of the tip bevel 152. A tip-bevel angle of the tip bevel 152 is greater than a primary-bevel angle of the primary bevel 154 such that the bevel provides a smooth transition over the needle tip 148. Such a needle tip is thusly configured for establishing a needle tract from an area of skin into a blood-vessel lumen of a patient in accordance with the needle tract-establishing step of the method set forth below.

The needle slot 150 extends from at least the proximal portion of the needle shaft 144 through the needle tip 148, thereby forming a needle channel 156 along at least a majority of a length of the needle shaft 144 as opposed to a needle lumen therethrough. That said, the needle slot 150 can extend from a proximal end of the needle shaft 144 through the needle tip 148, thereby forming the needle channel 156 along an entirety of the length of the needle shaft 144. The needle slot 150 has a width sized in accordance with an outer diameter of the access guidewire 106, which allows the access guidewire 106 to pass from the proximal portion of the needle shaft 144 through the needle tip 148 when the introducer needle-withdrawing step of the method set forth below is performed.

Notably, the needle shaft 144 includes the foregoing needle channel 156, whereas the introducer needle 104 includes a needle lumen 158. This is because the needle lumen 158 results from the combination of the needle shaft 144 and the sheath 142 over the needle shaft 144. Indeed, the sheath 142 over the needle shaft 144 seals the needle channel 156 forming the needle lumen 158 of the introducer needle 104 and enabling the syringe 112 to aspirate blood in accordance with the blood-aspirating step of the method set forth below.

The sheath 142 includes a sheath tip 160 in a distal portion of the sheath 142 and a sheath opening 162 in a side of the proximal portion of the sheath 142.

The sheath tip 160 includes a relatively short taper from an outer diameter of the distal portion of the sheath 142 to an outer diameter of a distal end of the sheath 142, the latter of which is commensurate with an outer diameter of the distal portion of the needle shaft 144. The taper has a taper angle less than the primary-bevel angle of the primary bevel 154 of the needle tip 148, which, in turn, is less than the tip-bevel angle of the tip bevel 152 of the needle tip 148. The sheath tip 160 including such a taper is configured to provide a smooth transition from the needle tip 148 to the sheath body for the needle tract-establishing step of the method set forth below.

The sheath opening 162 opens to the needle slot 150 of the needle shaft 144 allowing the access guidewire 106 to pass through the sheath opening 162 and into the needle slot 150 in the ready-to-operate state of the RICC insertion assembly 100. Thus, the sheath opening 162 has a width approximately commensurate with a width of the needle slot 150, which, in turn, is sized in accordance with the diameter of the access guidewire 106. The sheath opening 162 also has a length sufficient to allow the access guidewire 106 to pass through the sheath opening 162 and into the needle slot 150 while also accommodating the blade 212 of the splittable sealing module 196 under a distal end of the sheath opening 162. Notably, the sheath 142 over the needle shaft 144 seals the needle slot 150 thereunder except for that under the sheath opening 162. However, the splittable sealing module 196 seals over the needle slot 150 exposed by the sheath opening 162 by sealing the proximal portions of the needle shaft 144 and the sheath 142 therein, thereby enabling the syringe 112 to aspirate blood in accordance with the blood-aspirating step of the method set forth below.

The sheath 142 can be a cuttable or a splittable sheath configured for respectively cutting or splitting the sheath 142 away from the needle shaft 144 to allow the access guidewire 106 to escape from the needle shaft 144 by way of the needle slot 150. When configured to be cut away from the needle shaft 144, the sheath 142 can be formed of a polymeric material such as polyurethane that facilitates the cutting of the sheath 142 away from the needle shaft 144. When configured to be split away from the needle shaft 144, the sheath 142 can include a splitting means for splitting the sheath 142 that facilitates the splitting of the sheath 142 away from the needle shaft 144.

The splitting means for splitting the sheath 142 can include one or more weakened portions of the sheath 142 that facilitate the splitting of the sheath 142 away from the needle shaft 144. Indeed, as shown in FIG. 13B, the sheath 142 can be perforated with a pattern 228 of holes, slits, or a combination thereof longitudinally extending over the sheath 142 for the splitting of the sheath 142 away from the needle shaft 144. Notably, the pattern 228 is offset from the needle slot 150, thereby maintaining the seal provided by the sheath 142 over the needle shaft 144 and the needle slot 150 thereof despite the pattern 228 of holes, slits, or the combination thereof. Alternatively, as shown in FIG. 13C, the sheath 142 can include one or more grooves 230 longitudinally extending over the sheath 142. The sheath 142 is thinnest along the one-or-more grooves 230 allowing the splitting of the sheath 142 away from the needle shaft 144 along the one-or-more grooves.

The splitting means for splitting the sheath 142 can include an embedded pull cord 232 longitudinally extending within the sheath 142 that facilitates the splitting of the sheath 142 away from the needle shaft 144. Indeed, the pull cord 232 is configured to split the sheath 142 when the pull cord 232 is pulled away from the sheath 142. Optionally, the pull cord 232 includes a pull tab 234 over a proximal-end portion of the pull cord 232 for pulling the pull cord 232 away from the sheath 142. Whether or not the pull cord 232 includes the pull tab 234, the proximal-end portion of the pull cord 232 can extend from the coupler 108 in at least the ready-to-operate state of the RICC insertion assembly 100 for the pulling of the pull cord 232 away from the sheath 142 and along the coupler-housing slot 182 when needed for the splitting of the sheath 142 away from the needle shaft 144.

The sheath 142, or a sheath body thereof, is formed of a polymeric material configured to facilitate a smooth, consistent insertion of the introducer needle 104 from an area of skin to a blood-vessel lumen of a patient in accordance with the needle tract-establishing step of the method set forth below. In addition, the polymeric material has mechanical properties at a thickness of the sheath 142 sufficient to withstand collapse of the sheath 142 into the needle slot 150 of the needle shaft 144 when the blood-aspirating step of the method set forth below is performed, notably, while also facilitating the cutting or splitting of the sheath 142 off the needle shaft 144 in accordance with at least the introducer needle-withdrawing step of the method set forth below for the cutting of the sheath 142 off the needle shaft 144. Such a polymeric material can include, but is not limited to, polyethylene, polypropylene, polyurethane, or polytetrafluoroethylene (“PTFE”). In an example, the sheath 142 can be polyurethane in embodiments of the RICC insertion assembly 100 in which the sheath 142 is cut away from the needle shaft 144 by the blade 212. In another example, the sheath 142 can be PTFE or even expanded PTFE (“ePTFE”) in embodiments of the RICC insertion assembly 100 in which the sheath 142 is split away from the needle shaft 144. When the sheath 142 is, for example, ePTFE, the sheath 142 need not include the one-or-more weakened portions of the sheath 142 for the splitting of the sheath 142 away from the needle shaft 144 because ePTFE, itself, is the splitting means for splitting the sheath 142 on account of the longitudinal arrangement of polymer chains in the ePTFE.

The needle hub 146 includes an access-guidewire channel 164 in a distal portion of the needle hub 146 and a needle-hub connector 166 in a proximal portion of the needle hub 146.

The access-guidewire channel 164 of the needle hub 146 is configured to allow the access guidewire 106 to pass over the needle hub 146 and direct the access guidewire 106 into the access-guidewire passageway 206 of the splittable sealing module 196. The access-guidewire channel 164 is open such that the access guidewire 106 lies in the access-guidewire channel 164 in at least the ready-to-operate state of the RICC insertion assembly 100. Advantageously, the open access-guidewire channel 164 allows the access guidewire 106 to remain in place when the introducer needle 104 is withdrawn from the RICC insertion assembly 100 in accordance with the introducer needle-withdrawing step of the method set forth below.

Notably, the access-guidewire channel 164 of the needle hub 146 transitions into an access-guidewire channel 168 of the proximal portion 200 of the sealing-module insert 198 coupled to the distal portion of the needle hub 146. When the proximal portion 200 of the sealing-module insert 198 is combined with the distal portion 202 of the sealing-module insert 198, the access-guidewire channel 168 of the proximal portion 200 of the sealing-module insert 198 combines with that of the distal portion 202 of the sealing-module insert 198 to form the access-guidewire passageway 206 of the splittable sealing module 196. The needle hub 146 including the proximal portion 200 of the sealing-module insert 198 coupled thereto is configured to be removably disposed in the needle-hub receptacle 180 and the sealing-module cavity 178 of the coupler housing 172, respectively. As set forth below, the distal portion of the needle hub 146 axially compresses the proximal portion 200 of the sealing-module insert 198 in the sealing-module cavity 178 when the needle hub 146 is disposed in the needle-hub receptacle 180 and the proximal portion 200 of the sealing-module insert 198 is disposed in the sealing-module cavity 178. Axial compression of the proximal portion 200 of the sealing-module insert 198 in the sealing-module cavity 178, in turn, radially compresses both the proximal and distal portions 200 and 202 of the sealing-module insert 198 in the sealing-module cavity 178.

The needle-hub connector 166 includes a needle-hub bore 170 and an optional needle-hub flange (not shown) about the needle-hub connector 166.

The needle-hub bore 170 of the needle-hub connector 166 is configured to accept a syringe tip of the syringe 112 therein for fluidly connecting the introducer needle 104 to the syringe 112. Indeed, the needle-hub bore 170 can have a Luer taper (e.g., a 6% taper) configured to accept the syringe tip therein, which syringe tip can be complementarily configured with a Luer taper.

The needle-hub flange of the needle-hub connector 166 is configured to screw together with internal threads of a threaded collar around the syringe tip of the syringe 112. While the threaded collar of the syringe 112 is optional, the needle-hub flange advantageously provides a so-called Luer lock-style connection with the internal threads of the threaded collar when both are present. This provides added security against inadvertent disconnection of the introducer needle 104 and the syringe 112 over that provided by an otherwise Luer slip-style connection.

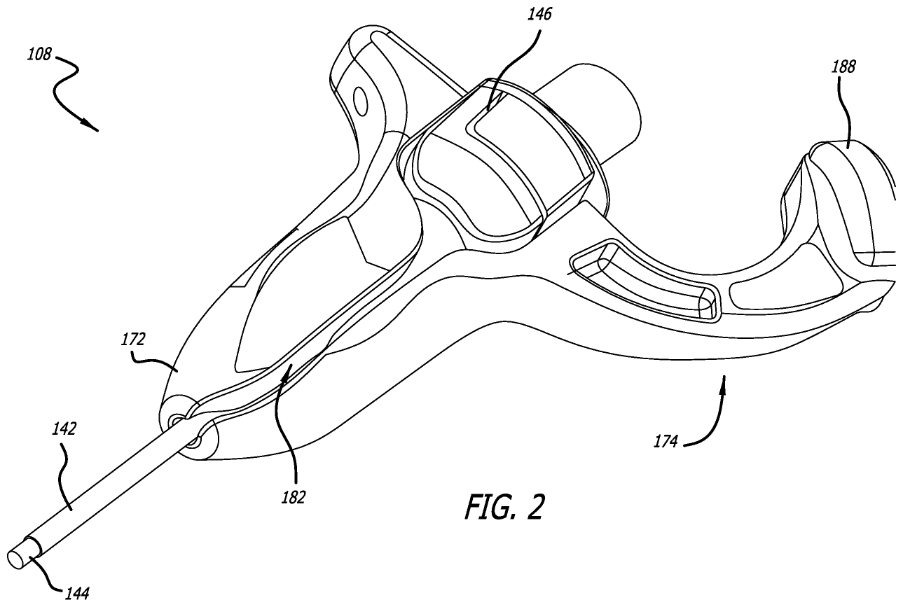

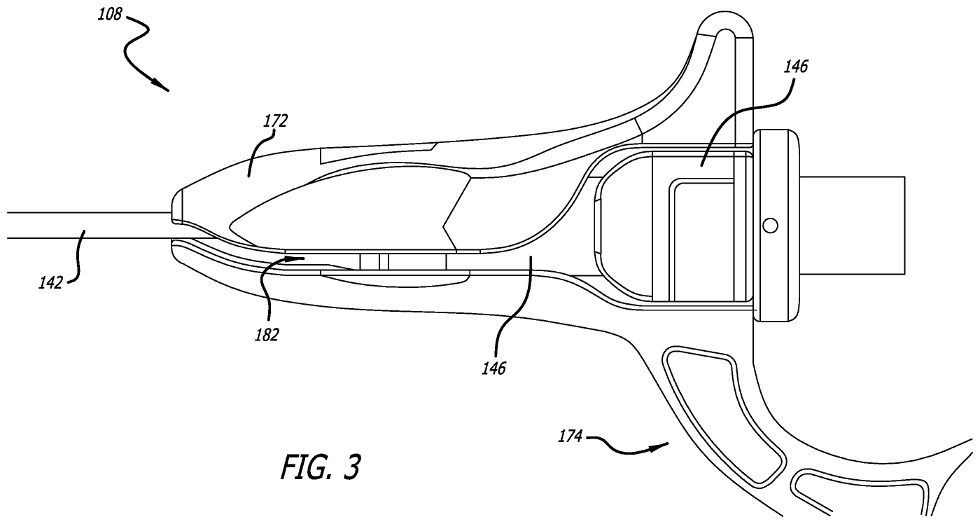

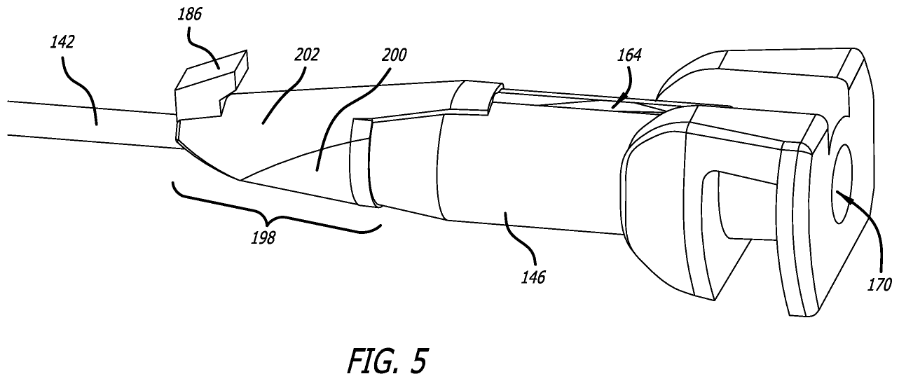

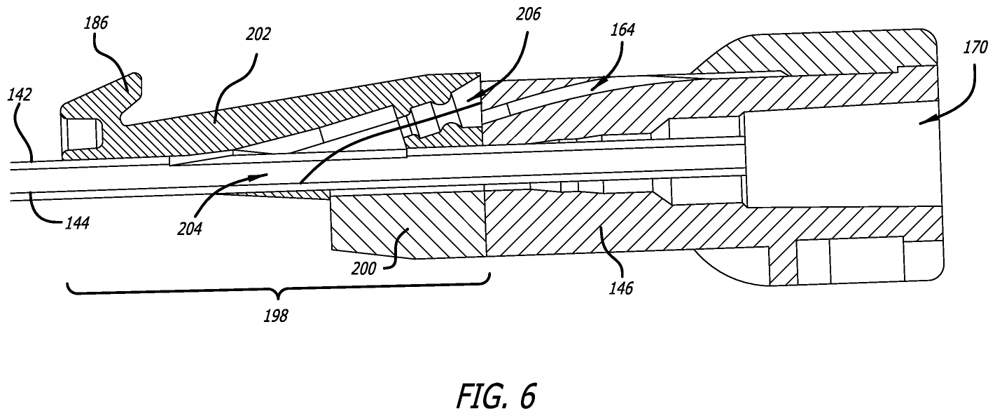

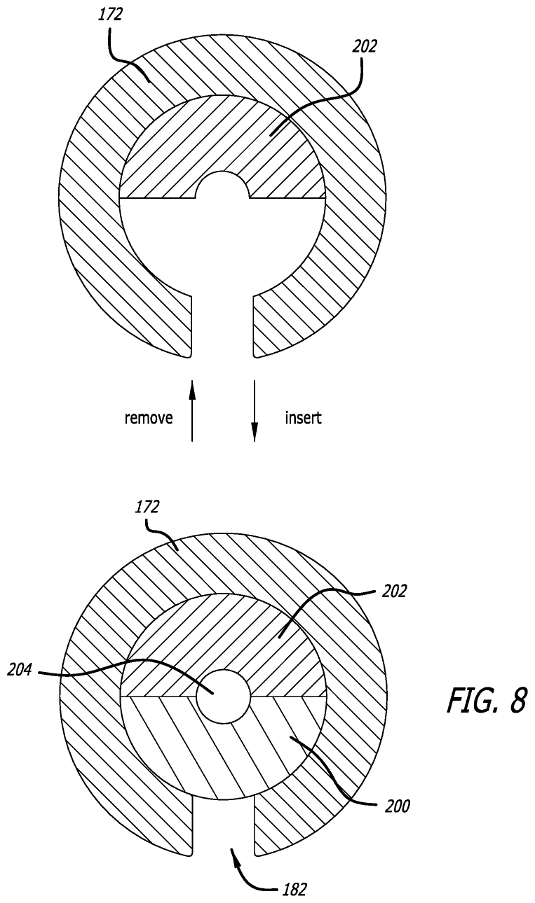

FIGS. 2-11 illustrate various view of the coupler 108 of the RICC insertion assembly 100 in accordance with some embodiments.

As shown, the coupler 108 includes a coupler housing 172, an access guidewire-connecting side arm 174, and, optionally, a splittable casing-holding side arm 176 when the RICC insertion assembly 100 also includes the keeper 220.

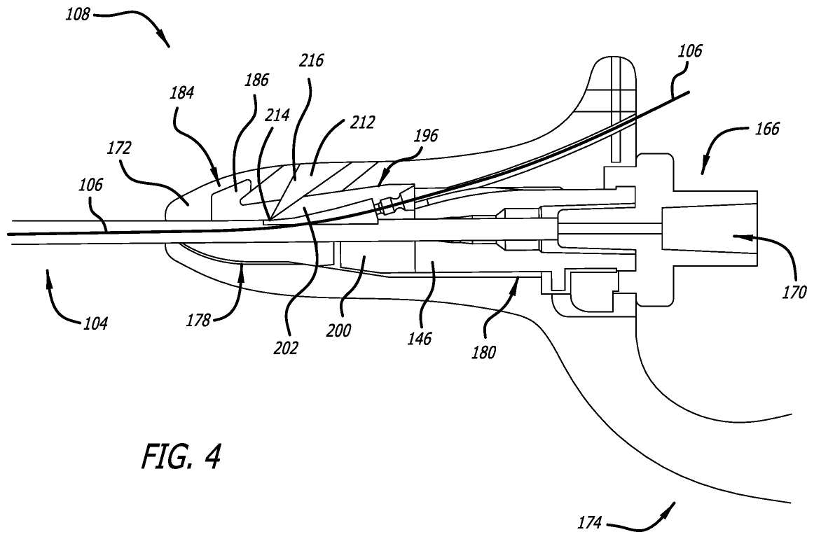

The coupler housing 172 includes a sealing-module cavity 178, a needle-hub receptacle 180 proximal of the sealing-module cavity 178, and a longitudinal coupler-housing slot 182 formed along a length of the coupler housing 172. (See FIG. 4, which includes the proximal and distal portions 200 and 202 of the sealing-module insert 198 disposed in the sealing-module cavity 178. FIG. 4 also includes the needle hub 146 of the introducer needle 104 disposed in the needle-hub receptacle 180.) Notably, the coupler housing 172 can be formed into a bullet-shaped body configured to be comfortably held underhand (e.g., cradled) or overhand in either a left hand for a left-handed venipuncture or a right hand for a right-handed venipuncture with the RICC insertion assembly 100. To further facilitate such venipunctures, an exterior of the coupler housing 172 can be textured with grip-enhancing ridges (e.g., transverse or circumferential ridges), protrusions, or the like.

The sealing-module cavity 178 is configured to hold the proximal and distal portions 200 and 202 of the sealing-module insert 198 therein. Indeed, the sealing-module cavity 178 includes at least the distal portion 202 of the sealing-module insert 198 captively disposed therein in each state of the one-or-more states of the RICC insertion assembly 100. For example, the sealing-module cavity 178 can include a catch 184 in a distal portion thereof, wherein the catch 184 is configured to accept therein a protrusion 186 of the distal portion 202 of the sealing-module insert 198 for captive disposal of the distal portion 202 of the sealing-module insert 198 in the sealing-module cavity. The sealing-module cavity 178 also includes the proximal portion 200 of the sealing-module insert 198 disposed therein in at least some states of the one-or-more states of the RICC insertion assembly 100 such as the ready-to-operate state or one or more operating states of the RICC insertion assembly 100.

The needle-hub receptacle 180 is configured to hold the needle hub 146 of the introducer needle 104 therein. Indeed, the needle-hub receptacle 180 includes the needle hub 146 inserted therein in the ready-to-operate state of the RICC insertion assembly 100. While not shown, the coupler 108 can include a needle-hub lock about the needle-hub receptacle 180 configured to lock the needle hub 146 in the needle-hub receptacle 180. Indeed, a pair of lock buttons (e.g., spring-loaded lock buttons) of the needle-hub lock can be distributed between opposite sides of the coupler housing 172 such that each lock button of the lock buttons extends through the coupler housing 172 on its respective side of the coupler 108. Such lock buttons can be configured to unlock the needle hub 146 when the lock buttons are pressed into the coupler housing 172 for withdrawal of the introducer needle 104 from the coupler 108. Unlocking the lock buttons can immediately release the axial compression from the distal portion of the needle hub 146 compressing the proximal portion 200 of the sealing-module insert 198 in the sealing-module cavity 178. This allows the proximal and distal portions 200 and 202 of the sealing-module insert 198 to relax for withdrawing the introducer needle 104 from the coupler 108. This also allows the proximal and distal portions of the sealing-module insert 198 to be separated for the escape of the access guidewire 106 when the introducer needle 104 is withdrawn from the coupler 108 in the introducer needle-withdrawing step of the method set forth below.

The coupler-housing slot 182 formed along the length of the coupler housing 172 is configured to allow the access guidewire 106 to escape from the coupler housing 172 after the introducer needle 104 is withdrawn from the coupler 108 in the introducer needle-withdrawing step of the method set forth below. Indeed, as the introducer needle 104 is withdrawn from the coupler 108 in the introducer needle-withdrawing step, the proximal portion 200 of the sealing-module insert 198, too, is withdrawn from the sealing-module cavity 178 allowing the access guidewire 106 to escape from the coupler housing 172 once the proximal portion 200 of the sealing-module insert 198 is removed from the sealing-module cavity 178.

The access guidewire-connecting side arm 174 extends from the coupler 108 or the coupler housing 172 thereof. The access guidewire-connecting side arm 174 includes a connector 188 configured to connect to the access-guidewire hub 218 about the proximal-end portion of the access guidewire 106, which access-guidewire hub 218 extends from the proximal end of the RICC 102 in at least the ready-to-operate state of the RICC insertion assembly 100. While in the ready-to-operate state of the RICC insertion assembly 100, the distal portion of the access guidewire 106 is disposed in the needle shaft 144 and the access-guidewire hub 218 is connected to the connector 188 of the access guidewire-connecting side arm 174, thereby enforcing the loop 110 in the access guidewire 106 over which loop 110 the RICC 102 is disposed. When both the keeper 220 and the splittable casing-holding side arm 176 are present in the RICC insertion assembly 100, a distal portion of the splittable casing 222 is also held in the primary channel 190 of the splittable casing-holding side arm 176, thereby further enforcing the loop 110 in the access guidewire 106 over which loop 110 the RICC 102 is disposed.

When present, the splittable casing-holding side arm 176 extends from the coupler 108 or the coupler housing 172 thereof opposite the access guidewire-connecting side arm 174. The splittable casing-holding side arm 176 includes a primary channel 190 and a secondary channel 192. The primary channel 190 is configured to slidably hold the splittable casing 222 or the longitudinal composite of the splittable casing 222 and at least the access guidewire 106 therein. The secondary channel 192 is configured to guide the access guidewire 106 split away from the splittable casing 222 into the coupler 108, the splittable sealing module 196 thereof, and the needle shaft 144 sealed therein by way of the needle slot 150. A divergent point 194 of the splittable casing-holding side arm 176 between the primary channel 190 and the secondary channel 192 is configured to split the access guidewire 106 away from the splittable casing 222 as the longitudinal composite of the splittable casing 222 and at least the access guidewire 106 therein is pushed therein.

FIGS. 4-11 illustrate various views of a splittable sealing module 196 in accordance with some embodiments.

The splittable sealing module 196 of the RICC insertion assembly 100 includes the sealing-module cavity 178 of the coupler housing 172 and an elastomeric sealing-module insert 198 disposed therein, which sealing-module insert 198 is split between a captive proximal portion 200 and a removable distal portion 202 referred to herein as the proximal portion 200 of the sealing-module insert 198 and the distal portion 202 of the sealing-module insert 198, respectively. (Notably, the proximal and distal portions 200 and 202 of the sealing-module insert 198 can be formed of a same elastomer [e.g., silicone] or different elastomers.) The proximal portion 200 of the sealing-module insert 198 is configured to be disposed in or otherwise inserted into the sealing-module cavity 178 of the coupler housing 172 where the proximal portion 200 of the sealing-module insert 198 combines with the distal portion 202 of the sealing-module insert 198 to complete a pair of passageways through the splittable sealing module 196. By way of the foregoing passageways, the splittable sealing module 196 is configured to separately seal around the introducer needle 104 and the access guidewire 106 when the proximal and distal portions 200 and 202 of the sealing-module insert 198 are compressed in the sealing-module cavity 178 in the one-or-more states of the RICC insertion assembly 100 (e.g., the ready-to-operate state or the one-or-more operating states of the RICC insertion assembly 100), which allows the syringe 112 is able to aspirate blood in accordance with the blood-aspirating step of the method set forth below.

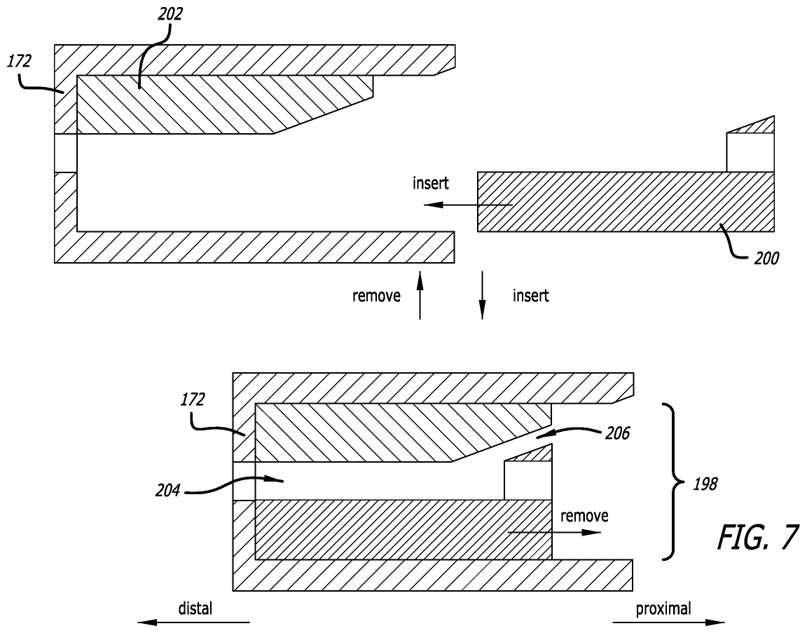

The pair of passageways completed when the proximal and distal portions 200 and 202 of the sealing-module insert 198 are combined in the sealing-module cavity 178 of the coupler housing 172 includes an introducer-needle passageway 204 and an access-guidewire passageway 206. As shown in FIG. 7, a proximal portion of the introducer-needle passageway 204 can be present in the proximal portion 200 of the sealing-module insert 198, but a distal portion of the introducer-needle passageway 204 is completed by the proximal and distal portions 200 and 202 of the sealing-module insert 198 when combined in the sealing-module cavity 178 of the coupler housing 172. As further shown in FIG. 7, an entirety of the access-guidewire passageway 206 can be completed by the proximal and distal portions 200 and 202 of the sealing-module insert 198 when combined in the sealing-module cavity 178 of the coupler housing 172. While other configurations of the sealing-module insert 198 are possible for providing the foregoing passageways, which configurations range from alternative configurations of the proximal and distal portions 200 and 202 of the sealing-module insert 198 to inclusion of one or more additional portions (e.g., a medial portion) of the sealing-module insert 198, the splittable sealing module 196 is configured by way of the introducer-needle passageway 204 and the access-guidewire passageway 206 to separately seal around the proximal portion of the introducer needle 104 and the distal portion of the access guidewire 106, respectively, when the proximal and distal portions 200 and 202 of the sealing-module insert 198 are compressed in the sealing-module cavity 178 in the one-or-more states of the RICC insertion assembly 100. Notably, as opposed to the introducer-needle passageway 204, which passes through both proximal and distal ends of the sealing-module insert 198, a distal end of the access-guidewire passageway 206 connects to a medial portion of the introducer-needle passageway 204, thereby allowing the distal portion of the access guidewire 106 to be disposed in the needle shaft 144 while the distal portion of the access guidewire 106 is disposed in the splittable sealing module 196. Indeed, the access-guidewire passageway 206 is configured to direct the access guidewire 106 from the access-guidewire channel 164 of the needle hub 146 into both the sheath opening 162 of the sheath 142 and the needle slot 150 of the needle shaft 144 thereunder such that the access guidewire 106 can be disposed in the needle shaft 144 with the distal end of the access guidewire 106 just proximal of the needle tip 148 in the ready-to-operate state of the RICC insertion assembly 100.

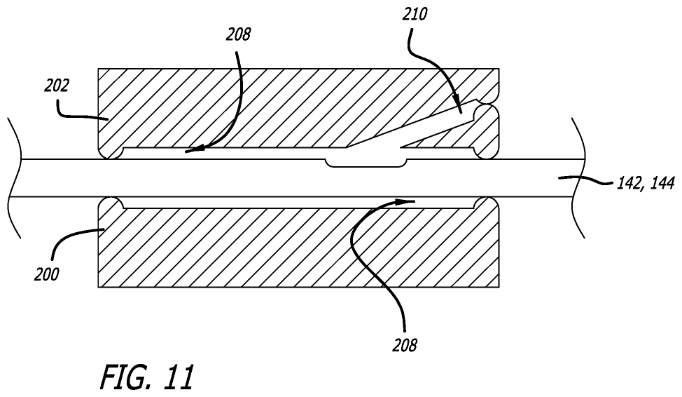

FIG. 11 illustrates a longitudinal cross section of the sealing-module insert 198 with internal reliefs 208 and 210 in the introducer-needle passageway 204 and the access-guidewire passageway 206 of the sealing-module insert 198 in accordance with some embodiments.

As shown, the introducer-needle passageway 204 formed between the proximal and distal portions 200 and 202 of the sealing-module insert 198 can include an internal relief 208 between proximal and distal portions of the introducer-needle passageway 204 to reduce friction on the introducer needle 104 when being withdrawn from the coupler 108 and the splittable sealing module 196 thereof in the introducer needle-withdrawing step of the method set forth below. Additionally or alternatively, the access-guidewire passageway 206 can include an internal relief 210 in at least a proximal portion of the access-guidewire passageway 206 to reduce friction on the access guidewire 106 when being advanced into the coupler 108 and the splittable sealing module 196 thereof in the access guidewire-advancing step of the method set forth below. With the internal relief 208 between proximal and distal portions of the introducer-needle passageway 204, only proximal- and distal-end portions of the introducer-needle passageway 204 including proximal and distal ends of the introducer-needle passageway 204 are configured to seal around the proximal portion of the introducer needle 104 when the proximal and distal portions 200 and 202 of the sealing-module insert 198 are compressed in the sealing-module cavity 178 of the coupler housing 172. With the internal relief 210 in the proximal portion of the access-guidewire passageway 206, only a proximal-end portion of the access-guidewire passageway 206 including a proximal end of the access-guidewire passageway 206 is configured to seal around the distal portion of the access guidewire 106 when the proximal and distal portions 200 and 202 of the sealing-module insert 198 are compressed in the sealing-module cavity 178 of the coupler housing 172. Notably, while the internal reliefs 208 and 210 are configured to respectively reduce friction of the introducer-needle passageway 204 and the access-guidewire passageway 206 on the introducer needle 104 and the access guidewire 106, respectively, a lubricant can be additionally or alternatively employed in the sealing-module insert 198 to reduce the friction.

The splittable sealing module 196 is configured to separately seal around the proximal portion of the introducer needle 104 and the distal portion of the access guidewire 106 when the sealing-module insert 198 is compressed in the sealing-module cavity 178. Indeed, the proximal and distal portions 200 and 202 of the sealing-module insert 198 can be radially compressed in the sealing-module cavity 178 or both axially and radially compressed in the sealing-module cavity 178 to seal around the introducer needle 104 and the access guidewire 106. With seals around the introducer needle 104 and the access guidewire 106 in accordance with the foregoing, the syringe 112 is able to aspirate blood in accordance with the blood-aspirating step of the method set forth below.

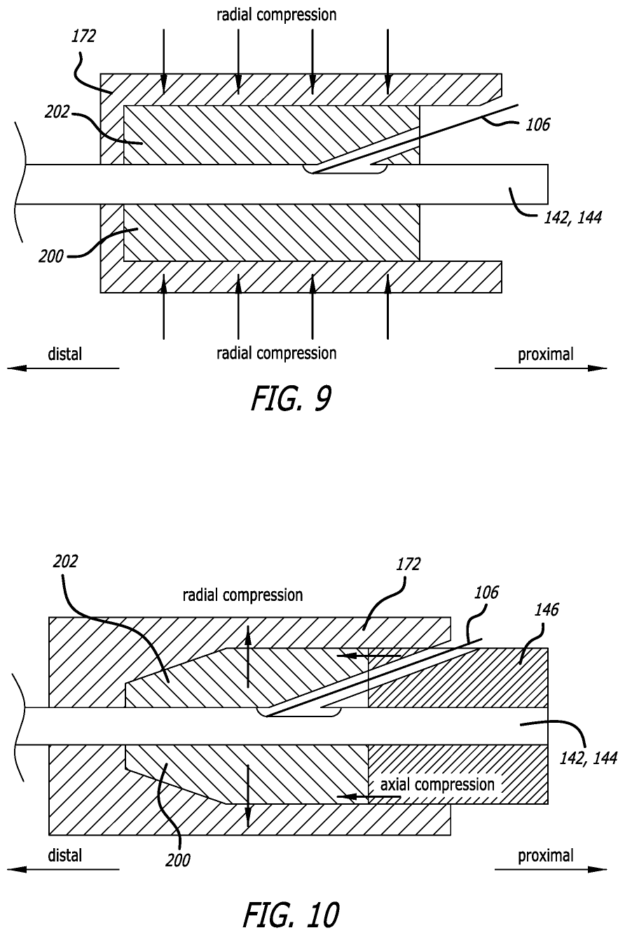

In an example of radial compression of the sealing-module insert 198 in the sealing-module cavity 178, the proximal and distal portions 200 and 202 of the sealing-module insert 198 can be radially compressed in the sealing-module cavity 178 by the coupler housing 172 itself as shown in FIG. 9. Without limiting such embodiments, two halves of the coupler housing 172 can be coupled together like a clamshell by way of a hinge opposite the coupler-housing slot 182 formed between the two halves of the coupler housing 172. One or more clamps across the coupler-housing slot 182 can be clamped in at least the ready-to-operate state of the RICC insertion assembly 100 to hold the two halves of the coupler housing 172 together, apply sufficient pressure to radially compress the proximal and distal portions 200 and 202 of the sealing-module insert 198 in the sealing-module cavity 178, and seal the sealing-module insert 198 around the introducer needle 104 and the access guidewire 106. The one-or-more clamps can be unclamped in the one-or-more operating states of the RICC insertion assembly 100 to relieve the radial compression and allow the introducer needle 104 to be withdrawn from the coupler 108 for the escape of the access guidewire 106 through the coupler-housing slot 182 thereafter.

In an example of both radial and axial compression of the sealing-module insert 198 in the sealing-module cavity 178, the proximal and distal portions 200 and 202 of the sealing-module insert 198 can be both axially and radially compressed in the sealing-module cavity 178 by way of the distal portion of the needle hub 146. The distal portion of the needle hub 146 axially compresses the proximal and distal portions 200 and 202 of the sealing-module insert 198 in the sealing-module cavity 178 when the needle hub 146 is disposed in the needle-hub receptacle 180 such as in the ready-to-operate state of the RICC insertion assembly 100. Axial compression of the proximal and distal portions 200 and 202 of the sealing-module insert 198 in the sealing-module cavity 178, in turn, radially compresses the proximal and distal portions 200 and 202 of the sealing-module insert 198 in the sealing-module cavity 178, thereby sealing the sealing-module insert 198 around the introducer needle 104 and the access guidewire 106. The needle hub 146 can be removed from the needle-hub receptable in the one-or-more operating states of the RICC insertion assembly 100 to relieve both the axial and the radial compression and allow the introducer needle 104 to be withdrawn from the coupler 108 for the escape of the access guidewire 106 through the coupler-housing slot 182 thereafter.

The coupler housing 172 of the coupler 108 can further include a blade 212 extending into the introducer-needle passageway 204 of the splittable sealing module 196. The blade 212 can be overmolded into the distal portion 202 of the sealing-module insert 198, thereby integrating the blade 212 therein. The blade 212 or a blade tip 214 thereof can be disposed in the needle slot 150 under a distal end of the sheath opening 162 in at least the ready-to-operate state of the RICC insertion assembly 100. The blade 212 includes a distal-facing blade edge 216 configured to cut the sheath 142 away from the needle shaft 144 as the introducer needle 104 is withdrawn from the coupler 108 through the introducer-needle passageway 204 in the introducer needle-withdrawing step of the method set forth below. Cutting the sheath 142 away from the needle shaft 144 as the introducer needle 104 is withdrawn from the coupler 108 allows the access guidewire 106 to escape from the needle shaft 144 by way of the needle slot 150.

FIGS. 1, 4, 9, and 10 illustrate various view of the access guidewire 106 of the RICC insertion assembly 100 in accordance with some embodiments.

The access guidewire 106 includes a proximal portion including a proximal end and a distal portion including a distal end. In the ready-to-operate state of the RICC insertion assembly 100, the proximal end of the access guidewire 106 is coupled to the access guidewire-connecting side arm 174 by way of an access-guidewire hub 218 about a proximal-end portion of the access guidewire 106 that includes the distal end. In addition, the proximal portion of the access guidewire 106 extends along the primary lumen 130 of the RICC 102. The distal portion of the access guidewire 106 also extends along the primary lumen 130 of the RICC 102, but the distal portion of the access guidewire 106 further extends out the distal end of the RICC 102 as an extracatheteral portion of the access guidewire 106, into the splittable sealing module 196 over the needle hub 146 by way of the access-guidewire channel 164, into the needle shaft 144 through both the sheath opening 162 of the sheath 142 and the needle slot 150 of the needle shaft 144, and along the needle lumen 158 of the introducer needle 104 in the ready-to-operate state of the RICC insertion assembly 100. As shown in FIG. 1, the distal end of the access guidewire 106 is disposed in the needle lumen 158 just proximal of the needle tip 148 in the ready-to-operate state of the RICC insertion assembly 100. Again, the proximal and distal ends of the access guidewire 106 enforce the loop 110 in the access guidewire 106 in the ready-to-operate state of the RICC insertion assembly 100, which loop 110 the RICC 102 is disposed over, thereby keeping the RICC insertion assembly 100 in a relatively compact form.

The access guidewire 106 can include a guidewire tip in the distal portion of the access guidewire 106, which adopts a T shape configured to prevent puncturing a back wall of a blood vessel. Such a guidewire tip assumes a straightened state in the ready-to-operate state of the RICC insertion assembly 100 and a curved state when the guidewire tip is advanced beyond the needle tip 148 (e.g., advanced into a blood-vessel lumen) in the one-or-more operating states of the RICC insertion assembly 100 in which the access guidewire 106 is deployed.

The access guidewire 106 can further include a bare-wire portion and a wound-wire portion distal of the bare-wire portion, proximal of the bare-wire portion, or both. While not shown, the bare-wire portion, when present, distally extends through the access-guidewire passageway 206 of the splittable sealing module 196 in at least the ready-to-operate state of the RICC insertion assembly 100 such that the splittable sealing module 196 forms a fluid-tight seal around the bare-wire portion of the access guidewire 106. Notably, the foregoing bare-wire portion can instead be a flat-wound or ground-wound portion of the access guidewire 106, wherein the flat-wound portion includes windings of a tape instead of a round wire, and wherein the ground-wound portion includes windings of a round wire ground down to flatten the windings.

FIG. 1 illustrate a keeper 220 of the RICC insertion assembly 100 in accordance with some embodiments.

As shown, the keeper 220 can include a splittable casing 222 and a catheter-hub holder 224 to which a proximal end of the splittable casing 222 is attached.

The splittable casing 222 can form a longitudinal composite with the catheter tube 114, the access guidewire 106, or both the catheter tube 114 and the access guidewire 106 in the RICC insertion assembly 100. With respect to the RICC insertion assembly 100 of FIG. 1, for example, the splittable casing 222 is over and, thusly, forms the longitudinal composite with both the catheter tube 114 and the access guidewire 106 in a portion of the RICC insertion assembly 100 nearest the catheter-hub holder 224, in which portion the access guidewire 106 is disposed in the primary lumen 130 of the RICC 102. Further with respect to the RICC insertion assembly 100 of FIG. 1, the splittable casing 222 is over and, thusly, forms the longitudinal composite with just the access guidewire 106 in a portion of the RICC insertion assembly 100 nearest the coupler 108 or the splittable casing-holding side arm 176 thereof, in which portion the extracatheteral portion of the access guidewire 106 extends from the distal end of the RICC 102. The splittable casing 222 is configured to split along its length, for example, as it is slid over the divergent point 194 of the splittable casing-holding side arm 176 of the coupler 108, such that the extracatheteral portion of the access guidewire 106 is initially revealed and the distal portion of the catheter tube 114 is subsequently revealed. In this way, the splittable casing 222 is configured to keep the catheter tube 114 and at least the distal portion of the access guidewire 106 sterile until deployed.

The catheter-hub holder 224 is configured to hold the catheter hub 116 therein as well as keep the splittable casing 222 in position over the catheter tube 114 and the access guidewire 106, particularly the extracatheteral portion of the access guidewire 106. The catheter-hub holder 224 includes a perimetrical wall 226 around at least a portion (e.g., a proximal portion) of a perimeter of the catheter-hub holder 224. The perimetrical wall 226 defines a recess into which the catheter hub 116 fits with an engineering fit (e.g., a clearance fit such as a running, sliding, or location fit or a transition fit such as a similar or fixed fit as classified by the International Organization for Standardization [“ISO”]) as well as one or more gaps for extension of the one-or-more extension legs 118 therethrough. Additionally or alternatively, the catheter-hub holder 224 can include a wing corresponding to a suture wing of the catheter hub 116. Such a wing can include posts configured to insert into suture-wing holes of the suture wing of the catheter hub 116 with an engineering fit.

### Methods

Methods of the RICC insertion assembly 100 include a method for inserting the RICC 102 into a blood-vessel lumen of a patient. Such a method includes one or more steps selected from an insertion assembly-obtaining step, a state-confirming step, a needle tract-establishing step, a blood-aspirating step, an access guidewire-advancing step, a sheath-splitting step, an introducer needle-withdrawing step, a RICC-advancing step, an access guidewire-withdrawing step, a maneuver guidewire-advancing step, another RICC-advancing step, and a maneuver guidewire-withdrawing step.

The insertion assembly-obtaining step includes obtaining the RICC insertion assembly 100, optionally, already in the ready-to-operate state thereof. As set forth above, the RICC insertion assembly 100 includes the RICC 102, the introducer needle 104, the access guidewire 106, and the coupler 108 coupling the RICC 102 and the introducer needle 104 together. In addition, the RICC insertion assembly 100 includes the splittable sealing module 196 formed between at least the sealing-module cavity 178 of the coupler housing 172 of the coupler 108, the distal portion 202 of the sealing-module insert 198 disposed in the sealing-module cavity 178, and the proximal portion 200 of the sealing-module insert 198 coupled to the distal portion of the needle hub 146 of the introducer needle 104. The splittable sealing module 196 separately seals around the proximal portion of the introducer needle 104 and the distal portion of the access guidewire 106 in at least the ready-to-operate state of the RICC insertion assembly 100. Within the splittable sealing module 196, the distal portion of the access guidewire 106 is disposed in the needle shaft 144 through the needle slot 150. This is accomplished through a connection between the distal end of the access-guidewire passageway 206 and the medial portion of the introducer-needle passageway 204 in the sealing-module insert 198.

The state-confirming step includes confirming the RICC insertion assembly 100 is in the ready-to-operate state thereof before the needle tract-establishing step. Such confirmation ensures the proximal and distal portions 200 and 202 of the sealing-module insert 198 are compressed in the sealing-module cavity 178 sealing the introducer-needle passageway 204 and the access-guidewire passageway 206 around the introducer needle 104 and access guidewire 106, respectively. Notably, if the RICC insertion assembly 100 is not in the ready-to-operate state upon obtaining it in the insertion assembly-obtaining step, the RICC insertion assembly 100 can be adjusted to put it in the ready-to-operate state for subsequent steps.

The needle tract-establishing step includes establishing a needle tract from an area of skin to the blood-vessel lumen with the introducer needle 104. The needle tract-establishing step can also include ensuring blood flashes back into the introducer needle 104 (e.g., the needle hub 146 of the introducer needle 104), the syringe 112 (e.g., the syringe tip, a barrel of the syringe 112, or both) fluidly connected to the introducer needle 104, or both the introducer needle 104 and the syringe 112, thereby confirming the needle tract extends into the blood-vessel lumen. To enhance blood flashback, a slight vacuum can be drawn with the syringe 112 while establishing the needle tract.

The blood-aspirating step includes aspirating blood with the syringe 112 to confirm the needle tract extends into the blood-vessel lumen before withdrawing the introducer needle 104 from the coupler 108 in the introducer needle-withdrawing step. Again, the sheath 142 over the needle shaft 144 seals the needle slot 150 of the needle shaft 144 thereunder. In particular, the sheath 142 seals the needle slot 150 outside of the splittable sealing module 196. The splittable sealing module 196, in turn, seals over the sheath opening 162 of the sheath 142, which sheath opening 162 allows the access guidewire 106 to pass from the access-guidewire passageway 206 formed between the of the proximal and distal portions 200 and 202 of the sealing-module insert 198 and into the needle shaft 144 by way of the needle slot 150. The splittable sealing module 196 also seals around the distal portion of the access guidewire 106. Such seals enable the syringe 112 to aspirate blood in the blood-aspirating step.

The access guidewire-advancing step includes advancing the distal portion of the access guidewire 106 through both the splittable sealing module 196 and the needle slot 150 of the needle shaft 144 such that the distal end of the access guidewire 106 is advanced from its initial location in the introducer needle 104 into the blood-vessel lumen. As set for the above, the initial location of the distal end of the access guidewire 106 is just proximal of the needle tip 148 when the RICC insertion assembly 100 is in the ready-to-operate state of the RICC insertion assembly 100.

When the RICC insertion assembly 100 does not include the keeper 220 and the splittable casing 222 over both the catheter tube 114 of the RICC 102 and the extracatheteral portion of the access guidewire 106, the access guidewire-advancing step includes pinching the extracatheteral portion of the access guidewire 106 extending from the distal end of the RICC 102 and pushing the access guidewire 106 into the needle shaft 144 via the splittable sealing module 196 of the coupler 108. Advancing the access guidewire 106 in this way reduces the loop 110 in the access guidewire 106 over which loop 110 the RICC 102 is disposed. As set forth above, the loop 110 is enforced by the distal portion of the access guidewire 106 disposed in the needle shaft 144 and the access-guidewire hub 218 of the access guidewire 106 connected to the connector 188 of the access guidewire-connecting side arm 174 of the coupler 108.

When the RICC insertion assembly 100 includes the keeper 220 and the splittable casing 222 over both the catheter tube 114 of the RICC 102 and the extracatheteral portion of the access guidewire 106, the access guidewire-advancing step includes pinching the splittable casing 222 over both the catheter tube 114 of the RICC 102 and the extracatheteral portion of the access guidewire 106 extending from the distal end of the RICC 102. The access guidewire-advancing step also includes pushing the splittable casing 222 into the primary channel 190 of the splittable casing-holding side arm 176 of the coupler 108 while pinching the splittable casing 222. With such pinching and pushing of the splittable casing 222 into the primary channel 190 of the splittable casing-holding side arm 176, the access guidewire 106 splits away from the splittable casing 222 over the divergent point 194 of the splittable casing-holding side arm 176 between the primary channel 190 and the secondary channel 192, into the secondary channel 192 of the splittable casing-holding side arm 176, and into the needle shaft 144 via the splittable sealing module 196 of the coupler 108. Advancing the access guidewire 106 in this way reduces the loop 110 in the access guidewire 106 over which loop 110 the RICC 102 and the splittable casing 222 are disposed. The loop 110 is enforced by at least the splittable casing 222 held in the primary channel 190 of the splittable casing-holding side arm 176 and the access-guidewire hub 218 of the access guidewire 106 connected to the connector 188 of the access guidewire-connecting side arm 174 of the coupler 108.

The introducer needle-withdrawing step includes withdrawing the introducer needle 104 from the coupler 108 before the RICC-advancing step, thereby leaving the access guidewire 106 in place in the blood-vessel lumen. The withdrawing of the introducer needle 104 from the coupler 108 removes the needle hub 146 of the introducer needle 104 from the needle-hub receptacle 180 and, with the needle hub 146, the proximal portion 200 of the sealing-module insert 198 from the sealing-module cavity 178, thereby splitting the proximal and distal portions 200 and 202 of the sealing-module insert 198 away from each other and unsealing the proximal portion of the introducer needle 104 and the distal portion of the access guidewire 106 so the access guidewire 106 is allowed to escape the splittable sealing module 196.

The introducer needle-withdrawing step can also include cutting the needle slot-sealing sheath 142 of the introducer needle 104 away from the needle shaft 144. As set forth above, the coupler housing 172 of the coupler 108 can include the blade 212 extending into the splittable sealing module 196 such that the blade 212 is disposed in the needle slot 150 under the distal end of the sheath opening 162 of the sheath 142 with the distal-facing blade edge 216 for the cutting of the sheath 142 away from the needle shaft 144. Should the coupler housing 172 not include the blade 212, the method can further include the sheath-splitting step, which includes splitting the sheath 142 in accordance with the splitting means for splitting the sheath 142 set forth above. The cutting or splitting of the sheath 142 away from the needle shaft 144 allows the access guidewire 106 to escape from the needle shaft 144 by way of the needle slot 150. In addition, the coupler-housing slot 182 of the coupler housing 172 allows the access guidewire 106 to escape from the coupler housing 172 after the withdrawing of the introducer needle 104 from the coupler 108.

The RICC-advancing step includes advancing the catheter tube 114 of the RICC 102 over the access guidewire 106 and into the blood-vessel lumen, thereby inserting the RICC 102 into the blood-vessel lumen.

The access guidewire-withdrawing step includes withdrawing the access guidewire 106 leaving the catheter tube 114 in place in the blood-vessel lumen.

The maneuver guidewire-advancing step includes advancing a maneuver guidewire into the blood-vessel lumen by way of the primary lumen 130 of the RICC 102 and to a lower ⅓ of an SVC of a heart of the patient.

The other RICC-advancing step includes advancing the catheter tube 114 farther into the blood-vessel lumen over the maneuver guidewire to the lower ⅓ of the SVC of the heart of the patient.

The maneuver guidewire-withdrawing step includes withdrawing the maneuver guidewire leaving the catheter tube 114 in place in the lower ⅓ of the SVC.

While some particular embodiments have been disclosed herein, and while the particular embodiments have been disclosed in some detail, it is not the intention for the particular embodiments to limit the scope of the concepts provided herein. Additional adaptations or modifications can appear to those of ordinary skill in the art, and, in broader aspects, these adaptations or modifications are encompassed as well. Accordingly, departures may be made from the particular embodiments disclosed herein without departing from the scope of the concepts provided herein.

1. A rapidly insertable central catheter (“RICC”) insertion assembly, comprising:
a RICC including a catheter tube and a primary lumen therethrough;
an introducer needle including:
a needle shaft including a longitudinal needle slot;
a needle hub around a proximal-end portion of the needle shaft; and
a proximal portion of a sealing-module insert coupled to a distal portion of the needle hub;
an access guidewire including a proximal portion disposed in the primary lumen of the RICC and a distal portion disposed in the needle shaft through the needle slot; and
a coupler coupling the RICC and the introducer needle together, the coupler including:
a coupler housing including a sealing-module cavity; and
a distal portion of the sealing-module insert disposed in the sealing-module cavity, the sealing-module cavity and the proximal and distal portions of the sealing-module insert forming a splittable sealing module of the RICC insertion assembly configured to separately seal around a proximal portion of the introducer needle and the distal portion of the access guidewire when the proximal and distal portions of the sealing-module insert are compressed in the sealing-module cavity.

2. The RICC insertion assembly according to claim 1, wherein each portion of the proximal and distal portions of the sealing-module insert is formed of an elastomer.

3. The RICC insertion assembly according to claim 1, wherein the proximal and distal portions of the sealing-module insert complete both an introducer-needle passageway and an access-guidewire passageway as combined in the sealing-module cavity.

4. The RICC insertion assembly according to claim 3, wherein a proximal portion of the introducer-needle passageway is present in the proximal portion of the sealing-module insert and a distal portion of the introducer-needle passageway is completed by the proximal and distal portions of the sealing-module insert combined in the sealing-module cavity.

5. The RICC insertion assembly according to claim 3, wherein an entirety of the access-guidewire passageway is completed by the proximal and distal portions of the sealing-module insert combined in the sealing-module cavity.

6. The RICC insertion assembly according to claim 3, wherein a distal end of the access-guidewire passageway connects to a medial portion of the introducer-needle passageway, thereby allowing the distal portion of the access guidewire to be disposed in the needle shaft through the needle slot.

7. The RICC insertion assembly according to claim 3, wherein the introducer-needle passageway is configured to seal around the proximal portion of the introducer needle and the access-guidewire passageway is configured to seal around the distal portion of the access guidewire when the proximal and distal portions of the sealing-module insert are compressed in the sealing-module cavity.

8. The RICC insertion assembly according to claim 3, wherein the introducer-needle passageway includes an internal relief between proximal and distal portions of the introducer-needle passageway such that only proximal- and distal-end portions of the introducer-needle passageway are configured to seal around the proximal portion of the introducer needle when the proximal and distal portions of the sealing-module insert are compressed in the sealing-module cavity.

9. The RICC insertion assembly according to claim 3, wherein the access-guidewire passageway includes an internal relief in at least a proximal portion of the access-guidewire passageway such that only a proximal-end portion of the access-guidewire passageway is configured to seal around the distal portion of the access guidewire when the proximal and distal portions of the sealing-module insert are compressed in the sealing-module cavity.

10. The RICC insertion assembly according to claim 1, wherein the sealing-module insert is radially compressed in the sealing-module cavity by the coupler housing.

11. The RICC insertion assembly according to claim 1, wherein the sealing-module insert is axially compressed in the sealing-module cavity by the distal portion of the needle hub, axial compression of the sealing-module insert in the sealing-module cavity, in turn, radially compressing the sealing-module insert in the sealing-module cavity.

12. The RICC insertion assembly according to claim 1, wherein the coupler housing includes a longitudinal coupler-housing slot configured to allow the access guidewire to escape from the coupler housing after the proximal portion of the sealing-module insert is removed from the sealing-module cavity with withdrawal of the introducer needle from the coupler.

13. The RICC insertion assembly according to claim 1, the introducer needle further including a sheath over the needle shaft sealing the needle slot thereunder, the needle slot extending from a proximal portion of the needle shaft through a distal needle tip.

14. The RICC insertion assembly according to claim 13, wherein the coupler includes a blade extending into the splittable sealing module such that the blade is disposed in the needle slot under a distal end of a sheath opening, the blade including a distal-facing blade edge configured to cut the sheath away from the needle shaft as the introducer needle is withdrawn from the coupler, thereby allowing the access guidewire to escape from the needle shaft by way of the needle slot.

15. The RICC insertion assembly according to claim 14, wherein the blade is overmolded into the coupler housing, thereby integrating the blade in the coupler housing.

16. The RICC insertion assembly according to claim 13, wherein the sheath is splittable.

17. The RICC insertion assembly according to claim 16, wherein the sheath is perforated with a pattern of holes, slits, or a combination thereof longitudinally extending over the sheath configured for splitting the sheath, the pattern offset from the needle slot to maintain the sealing of the needle slot by the sheath.

18. The RICC insertion assembly according to claim 16, wherein the sheath includes one or more grooves longitudinally extending over the sheath configured for splitting the sheath.

19. The RICC insertion assembly according to claim 16, wherein the sheath includes an embedded pull cord longitudinally extending within the sheath configured for splitting the sheath when pulled away from the sheath.

20. The RICC insertion assembly according to claim 13, wherein the sheath is formed of a polymeric material selected from polyethylene, polypropylene, polyurethane, and polytetrafluoroethylene.

21. The RICC insertion assembly according to claim 1, the coupler further including an access guidewire-connecting side arm including a connector configured to connect to an access-guidewire hub about a proximal-end portion of the access guidewire, the distal portion of the access guidewire disposed in the needle shaft and the access-guidewire hub connected to the connector of the access guidewire-connecting side arm enforcing a loop in the access guidewire over which loop the RICC is disposed in at least a ready-to-operate state of the RICC insertion assembly.

22. The RICC insertion assembly according to claim 1, further comprising a keeper including a splittable casing over both the catheter tube of the RICC and an extracatheteral portion of the access guidewire extending from a distal end of the RICC, the splittable casing configured to keep the catheter tube and the extracatheteral portion of the access guidewire sterile until deployed.

23. The RICC insertion assembly according to claim 22, wherein the keeper further includes a catheter-hub holder to which a proximal end of the splittable casing is attached, the catheter-hub holder configured to hold a catheter hub of the RICC therein and keep the splittable casing in position over both the catheter tube and the extracatheteral portion of the access guidewire.

24. The RICC insertion assembly according to claim 23, wherein the catheter-hub holder includes a perimetrical wall around at least a portion of a perimeter of the catheter-hub holder, the perimetrical wall defining a recess into which the catheter hub fits with an engineering fit.

25. The RICC insertion assembly according to claim 22, the coupler further including a splittable casing-holding side arm including a primary channel and a secondary channel, the primary channel configured to slidably hold the splittable casing therein and the secondary channel configured to guide the access guidewire split away from the splittable casing into the coupler and the needle shaft through the needle slot.

26. The RICC insertion assembly according to claim 25, the coupler further including an access guidewire-connecting side arm including a connector configured to connect to an access-guidewire hub about a proximal-end portion of the access guidewire, the distal portion of the access guidewire disposed in the needle shaft, a distal portion of the splittable casing held in the primary channel of the splittable casing-holding side arm, and the access-guidewire hub connected to the connector of the access guidewire-connecting side arm enforcing a loop in the access guidewire over which loop the RICC is disposed in at least a ready-to-operate state of the RICC insertion assembly.

27. The RICC insertion assembly according to claim 1, wherein the RICC includes a set of three lumens including the primary lumen, a secondary lumen, and a tertiary lumen formed of fluidly connected portions of three catheter-tube lumens, three catheter-hub lumens, and three extension-leg lumens.

28. The RICC insertion assembly according to claim 27, wherein the primary lumen has a primary-lumen aperture in a distal end of the catheter tube, the secondary lumen has a secondary-lumen aperture in a side of a distal portion of catheter tube, and the tertiary lumen has a tertiary-lumen aperture in the side of the distal portion of the catheter tube proximal of the secondary-lumen aperture.

29-46. (canceled)
